(19) United States
(12) Patent Application Publication
(10) Pub. No.: US 2009/0260095 A1
(43) Pub. Date: Oct. 15, 2009

(54) Gene Marker for Evaluating Genetic Ability for Carcass Weight in Bovine and Method for Evaluating Genetic Ability for Carcass Weight Using the Same

(76) Inventors: Akiko Takasuga, Fukushima (JP); Toshio Watanabe, Fukushima (JP); Takashi Hirano, Fukushima (JP); Kouji Setoguchi, Kagoshima (JP); Tomoko Nagao, Kumamoto (JP); Masako Furuta, Kumamoto (JP); Toshiaki Oe, Tottori (JP); Kazuya Inoue, Miyazaki (JP)

Correspondence Address: BARNES & THORNBURG LLP, 11 SOUTH MERIDIAN, INDIANAPOLIS, IN, 46204, US

(21) Appl. No.: 12/415,143

(22) Filed: Mar. 31, 2009

(30) Foreign Application Priority Data
Mar. 31, 2008 (JP) 2008-91328

(51) Int. Cl. A01K 67/027; C12N 15/87; C12Q 1/68; C07K 14/00; C07H 21/04; C12N 15/00; C12N 15/74

(52) U.S. Cl. 800/15; 800/21; 435/6; 536/23.5; 530/350; 435/320.1

(57) ABSTRACT

The object of this invention is to provide a method for evaluating genetic ability for carcass weight in a bovine individual by using gene markers. According to the method, the nucleotide at the e9 site of the bovine NCAPG gene is determined. When it is G, genetic ability for increasing carcass weight is judged to be higher. Alternatively, the amino acid at the E9 site of the bovine NCAPG gene is determined. When it is methionine, genetic ability for increasing carcass weight is judged to be higher.

### CROSS-REFERENCE TO RELATED APPLICATIONS

This application claims the benefit of priority to Japan Patent Application No. 2008-91328, filed on Mar. 31, 2008, which is incorporated herein by reference.

### FIELD OF THE INVENTION

The present invention relates to gene markers for evaluating carcass weight in bovine and methods for evaluating carcass weight using the same.

### BACKGROUND OF THE INVENTION

Meat quality and carcass weight of beef cattle are economic traits directly linking to prices. To examine how to evaluate hereditary ability in association with these traits and how to use it for the improvement of cattle, methods such as one based on breeding values have been invented and developed.

Meat quality and carcass weight are considered to be quantitative traits involved in a plurality of genes. If genes or genomic regions, i.e., quantitative trait loci (QTL), which relatively greatly affect meat quality or carcass weight, can be identified and superior genotypes can be selected, such data could be utilized to improve cattle.

To date, by the QTL analyses using paternal half-sib families of Japanese Black (Wagyu) cattle, it has been reported that genomic regions affecting body weight or carcass weight are present on bovine chromosome 6 (Takasuga et al. (2007) Mamm. Genome 18, 125-136). Later, in another family of Japanese Black cattle, QTL for carcass weight was found in the identical regions on chromosome 6 (The Book of Abstracts for the 2nd Annual Meeting of Japanese Society of Animal Breeding and Genetics). Meanwhile, also in a Japanese Brown bull and its male offspring that has inherited its superior genetic traits, QTL for carcass weight were detected in almost the identical regions to those described above.

However, since it was not known what kind of genetic variation is actually responsible for the superior genetic trait, it was difficult to utilize the information for breeding or producing cattle.

Thus, an object of the present invention is to provide methods for evaluating genetic ability for carcass weight in a bovine individual by using gene markers.

### SUMMARY OF THE INVENTION

By analyzing genomic regions affecting body weight or carcass weight on bovine chromosome 6, the inventors found that, among SNPs in the NCAPG gene, the SNP located at the e9 site is the causative SNP or the SNP in linkage disequilibrium with the causative SNP for the QTL for body weight or carcass weight on bovine chromosome 6. Based on this finding, the inventors discovered that isolated DNA that contains the e9 site of the NCAPG gene and has guanine (G) as the nucleotide at the e9 site is useful as a gene marker for increasing carcass weight. Further, they revealed that the SNP of G at the e9 site is a dominant mutation and that the NCAPG gene containing this SNP encodes a mutated NCAPG protein in which the amino acid at the E9 site is methionine.

Thus, an embodiment of the present invention is the method for evaluating genetic ability for carcass weight in a bovine individual includes determining the nucleotide at the e9 site of the NCAPG gene or the amino acid at the E9 site of the NCAPG protein.

Further, another embodiment is the bovine NCAPGgene that has G at the e9 site or the bovine NCAPG protein that has methionine at the E9 site.

Further, another embodiment is an isolated DNA that contains a part or the whole of the bovine NCAPG gene containing the e9 site of the bovine NCAPG gene. In this DNA, the nucleotide at this e9 site is preferably G.

Further, another embodiment is the gene marker used to evaluate genetic ability for carcass weight in a bovine individual being an isolated DNA containing a part or the whole of the bovine NCAPG gene that contains the e9 site of the bovine NCAPG gene.

Another embodiment of the present invention is the method for selecting a bovine individual having higher genetic ability for carcass weight including steps of determining the nucleotide at the e9 site of the NCAPG gene in each bovine individual and selecting an individual in which the nucleotide is G in at least one of the alleles of the NCAPG gene.

Another embodiment is the method for increasing carcass weight of a bovine individual by changing the nucleotide at the e9 site to G in at least one of the alleles of the NCAPG gene or expressing the NCAPG protein in which the amino acid at the E9 site is methionine using gene recombination technology rather than crossbreeding.

Another embodiment is the bovine individual having an exogenous DNA encoding the NCAPG protein in which the amino acid at the E9 site is methionine. This exogenous DNA may be an expression vector expressing the NCAPG protein.

### DETAILED DESCRIPTION OF PREFERRED EMBODIMENTS

Embodiments of the present invention accomplished based on the above-described findings are hereinafter described in detail by giving Examples. Unless otherwise explained, methods described in standard sets of protocols such as J. Sambrook and E. F. Fritsch & T. Maniatis (Ed.), “Molecular Cloning, a Laboratory Manual (3rd edition), Cold Spring Harbor Press and Cold Spring Harbor, N.Y. (2001); and F. M. Ausubel, R. Brent, R. E. Kingston, D. D. Moore, J. G. Seidman, J. A. Smith, and K. Struhl (Ed.), “Current Protocols in Molecular Biology,” John Wiley & Sons Ltd., or alternatively, modified/changed methods from these are used. When using commercial reagent kits and measuring apparatus, unless otherwise explained, attached protocols to them are used.

The objective, characteristics, and advantages of the present invention as well as the idea thereof will be apparent to those skilled in the art from the descriptions given herein. It is to be understood that the embodiments and specific examples of the invention described hereinbelow are to be taken as preferred examples of the present invention. These descriptions are for illustrative and explanatory purposes only and are not intended to restrict the invention to these embodiments or examples. It is further apparent to those skilled in the art that various changes and modifications may be made based on the descriptions given herein within the intent and scope of the present invention disclosed herein.

### ==SNPs in the Bovine NCAPG Gene==

The nucleotide at the e9 site in the wild-type bovine NCAPG gene is T. However, as will be shown in the Example, when the nucleotide at the e9 site in the bovine NCAPG gene is G, carcass weight increases. Therefore, by determining the nucleotide at the e9 site among SNPs in the bovine NCAPG gene, carcass weight can be evaluated and/or predicted.

The e9 site as used herein refers to the nucleotide at position 1372 in cDNA (NM_{—}001102376) of the bovine NCAPG gene shown in SEQ ID NO: 1 as well as to any nucleotide corresponding to this nucleotide in the NCAPG gene on the bovine genome, NCAPG gene homologues, hnRNA and mRNA of the NCAPG genes etc.

The amino acid at the E9 site in the bovine wild-type NCAPG protein is isoleucine, whereas the bovine NCAPG gene in which the nucleotide at the e9 site is G encodes an NCAPG protein in which the amino acid at the E9 site is methionine. Therefore, in place of the nucleotide at the e9 site in an NCAPG gene, the amino acid at the E9 site in the bovine NCAPG protein may be determined.

The E9 site as used herein refers to the amino acid at position 442 in the bovine NCAPG protein (NP_{—}001095846) shown in SEQ ID NO: 2 as well as to any amino acid corresponding to this amino acid in partial peptides, NCAPG homologues, etc.

### ==Gene Marker==

The diagnostic marker as used herein designed for the evaluation of genetic ability for carcass weight in bovine individuals refers to a gene-related substance for detecting the SNP at the e9 site in the bovine NCAPG gene. Examples of the diagnostic marker include DNA containing the NCAPG gene, such as cDNA; hnRNA and mRNA, which are transcripts; a peptide, which is a translation product; a protein, which is the end product of gene expression; etc.

When a diagnostic marker is an isolated DNA such as genomic DNA or synthesized DNA such as cDNA, carrying the NCAPG gene etc., the nucleotide at the SNP may be determined in order to detect the SNP. Specifically, the nucleotide sequence may be directly determined; or alternatively, PCR or RFLPs may be used. The method for the detection is not particularly limited. Likewise, when a diagnostic marker is hnRNA or mRNA, which is a transcript of the NCAPG gene, the SNP can be detected by determining the RNA sequence. When the SNP is directly detected, the nucleic acid whose sequence is to be determined is not required to contain the NCAPG gene as a whole but may contain a part of the NCAPG gene or cDNA, at least the nucleotide containing the SNP at the e9 site, which can be determined.

When a diagnostic marker is an isolated peptide such as the NCAPG protein etc., the amino acid carrying a mutation may be directly determined by the conventional method to detect the previously mentioned mutation. When this mutation is directly detected, the peptide is not required to contain the NCAPG protein as a whole but may contain a part of the NCAPG protein, at least the amino acid at the e9 site containing the mutation, which can be determined.

### ==Method for Interpreting SNPs==

The type of the nucleotide at the e9 site may be molecular-biologically determined. For example, genomic DNA is extracted from bovine cells and the nucleotide at the e9 site of the genomic DNA is determined by the conventional method. When a bovine individual is homozygous or heterozygous for G at the nucleotide of the e9 site, it can be judged to have higher genetic ability for carcass weight.

Likewise, the amino acid at the E9 site can be determined by, for example, purifying NCAPG protein from bovine cells using an antibody or the like, and determining the amino acid sequence according to the conventional method. When the amino acid at this E9 site is methionine, the individual can be judged to have higher genetic ability for carcass weight.

Further, by using this evaluation method, bovine individuals having higher genetic ability for carcass weight can be selected from large numbers of cattle. That is, by determining the nucleotide at the e9 site of the NCAPG gene and selecting an individual in which the nucleotide is G in one of the alleles, or alternatively, by determining the amino acid at the E9 site of the NCAPG protein and selecting an individual in which the amino acid is methionine, a bovine individual having higher genetic ability for carcass weight can be selected.

It should be noted that since the NCAPG gene is highly conserved in cattle, the breeds of the cattle suitable for practice of the present invention include, but not particularly limited to, Japanese black cattle, Japanese Brown cattle, Holstein, etc.

### ==Artificial Manipulation of SNPs==

In the bovine individuals in which the nucleotide is G at the e9 site in at least one of the alleles of the NCAPG gene and which express an NCAPG protein in which the amino acid at the E9 site is methionine, carcass weight increases, as will be described in the Example. In the NCAPG gene, no mutation has occurred at any site other than the e9 site; or, if at all, it is not associated with carcass weight.

Therefore, in order to increase carcass weight of bovine individuals, not by crossbreeding but by widely-known gene recombination methods such as, generation of knockout animals, knockdown animals, transgenic animals etc., individuals in which the nucleotide at the e9 site is substituted by G in at least one of the alleles of the NCAPG gene, or individuals expressing an NCAPG protein in which the amino acid at the E9 site is methionine may be generated.

To date, embryonic stem cells have been established using cattle (Biochem.Biophys.Res.Commun.vol. 309, p. 104-113, 2003), and knockout cattle have been generated as well (Nat Genet vol. 36, p. 671-672, 2004). By using gene recombination technology combined with developmental engineering, it is also possible to substitute nucleotides of interest for specific nucleotides in bovine individuals.

Thus, to increase carcass weight of bovine individuals having G as the nucleotide at the e9 site in neither of the alleles of the NCAPG gene, for example, individuals in which the nucleotide is substituted by G in at least one of the alleles of the NCAPG gene may be generated. In this generation, since this G-allele is dominant, both alleles should not necessarily be substituted: only one allele is sufficient to be substituted.

Alternatively, as will be described in the Example, since this is a dominant mutation, bovine individuals with increased carcass weight can be produced by genetically engineering cattle expressing a mutated NCAPG protein in which the amino acid at the E9 site is methionine. Specifically, for example, transgenic cattle into which an expression vector expressing the mutated protein has been introduced may be generated.

### Example

Hereinafter, the present invention will be explained in more detail with reference to Examples.

### (1) Methods for Extracting DNA and Genotyping Microsatellites and SNPs

Genomic DNA was extracted from semen, adipose tissues around the kidney, or blood by the conventional method. Genomic regions were amplified by the PCR method using primers with which genomic fragments of interest can be specifically amplified.

Microsatellites were genotyped by PCR amplification using forward and fluorescent-labeled reverse primers, followed by electrophoresis using ABI 3730 DNA analyzer (Applied Biosystems) and analysis using GENESCAN and GeneMapper software (Applied Biosystems). SNPs were detected and genotyped by direct sequencing of PCR products using Big Dye Terminator v.3.1 Cycle Sequencing Kit (Applied Biosystems). Since SNP 19 shown in Table 2 was a tandem repeat polymorphism, it was genotyped in the same way used for microsatellites.

### (2) Method for Measuring Carcass Weight

Carcass weight was measured based on carcass grading data of beef cattle at the slaughterhouses.

### (3) Statistical Analysis

In this Example, it is shown that the G-allele of the e9 site in the NCAPG gene is a dominant or additive mutation, affecting carcass weight.

Genomic DNAs of 3 Japanese Black sires (A-C) and2 Japanese Brown sires (D, E), in which QTL for carcass weight or bodyweight had been detected on bovine chromosome 6, were genotyped and compared using a large number of microsatellite markers and SNP markers generated using the bovine genome sequences. In this analysis, in order to determine the phase of the sire's chromosomes, offspring of each sire were also genotyped. The primers used are shown in Table 1.

TABLE 1

| | | Forward primer | Reverse primer | |
|---|---|---|---|---|
| cM | marker | (Seq ID No.) | (Seq ID No.) | base |
| | DIK9014 | AGCCAGCTGAGTCAAATTCC(3) | GTGAGACAGATGGGCAATCA(4) | 37,780,130 |
| 45.93 | DIK4852 | TCAGCTTCTGTACCCATGGAC(5) | AGCCAGGGTTTCCAGAAAAG(6) | 37,855,588 |
| | SNP 0 | CACCATGTCCTGACCTCAGAT(53) | TAACAGTGCCCTGCATGAGA(54) | 38,009,206 |
| | DIK9015 | CCTTTGTTTGCTGGGTCAAT(7) | GGGCTTGATCTCTGGTTGAG(8) | 38,051,344 |
| | DIK9016 | ATGGCAACCCACTACTCCAG(9) | TTGCTACCAAGCAAGCACTG(10) | 38,162,665 |
| | DIK9017 | GTAAACTCAAGCCACGGCA(11) | CGACAACCTTGATGTGACAAA(12) | 38,670,448 |
| | DIK9018 | GATGGCACTGGAGGTAGAGC(13) | CAACCCCATGGATTGTAACC(14) | 38,948,770 |

cM: Position on the linkage map (Ihara et al. (2004) Genome Res. 14, 1987-1998.)
base: Position on bovine chromosome 6 denoted by the number of the first nucleotide of the primer in the bovine genome sequence (2007-Sep-13) (http://www.hgsc.bcm.tmo.edu/).
Base of SNP 0 denotes the position of the SNP

The results revealed that the region spanning approximately 660 kb (SNP0-DIK9017) containing the NCAPG gene was common among the superior alleles of 5 sires and contained markers that distinguish the superior alleles from the inferior alleles in all the 5 sires.

The coding regions of 4 genes present in the 660 kb region were screened for SNPs. As a result, 5 SNPs which were heterozygous in Sire A and accompanying an amino acid substitution were identified. Examinations of these 5 SNPs in 5 sires revealed that only the SNP at the e9 site was heterozygous in all the 5 sires.

Nineteen adjacent SNPs (Table 2) including this SNP were examined for the effect on carcass weight.

TABLE 2

| | | Nucleotide | | | | a.a. | |
|---|---|---|---|---|---|---|---|
| | | of the | Sire A | | | mutation | |
| SNP ID | Base | sense DNA | (Q/q) | Gene | Region | from q to Q | MAF |
| SNP 1 | 38055058 | C | G/C | LOC523874 | intron | — | 0.42 |
| | | | | | (exon 5-6) | | |
| SNP 2 | 38055970 | A | G/A | | exon 4 | Lys→Glu | 0.42 |
| SNP 3 | 38058985 | G | A/G | | exon 2 | no change | 0.43 |
| SNP 4 | 38121891 | C | G/C | | exon 1 | Ala→Gly | 0.24 |
| SNP 5 | 38157198 | G | A | NCAPG | exon 4 | no change | 0.32 |
| SNP 6 | 38157668 | T | TTT | | intron | — | 0.32 |
| | | | | | (exon 5-6) | | |
| SNP 7 | 38163729 | A | G | | exon 8 | no change | 0.32 |
| SNP 8 | 38164388 | A | C | | exon 9 | no change | 0.32 |
| SNP 9 | 38164403 | T | G/T | | exon 9 | Ile→Met | 0.14 |
| SNP 10 | 38166283 | C | A/C | | intron | — | 0.24 |
| | | | | | (exon 9-10) | | |
| SNP 11 | 38166304 | T | T/A | | intron | — | 0.44 |
| | | | | | (axon 9-10) | | |
| SNP 12 | 38166927 | T | T/C | | intron | — | 0.45 |
| | | | | | (exon 11-12) | | |
| SNP 13 | 38180790 | T | C | | exon 14 | no change | 0.32 |
| SNP 14 | 38195339 | C | A/C | | exon 17 | Leu→Met | 0.24 |
| SNP 15 | 38195743 | A | G | | intron | — | 0.32 |
| | | | | | (exon 18-19) | | |
| SNP 16 | 38196233 | T | TT/T | | intron | — | 0.13 |
| | | | | | (exon 19-20) | | |
| SNP 17 | 38198882 | G | A | | intron | — | 0.32 |
| | | | | | (exon 20-21) | | |
| SNP 18 | 38231068 | C | C/T | LOC540095 | 3′UTR | — | 0.44 |
| SNP 19 | 38378214-31 | (GCC)6 | (GCC)6/7 | | exon 1 | Ala | 0.44 |
| | | | | | | deletion | |

base: Position on bovine chromosome 6 denoted by the number of the first nucleotide of the primer in the bovine genome sequence (2007-Sep-13) (http://www.hgsc.bcm.tmc.edu/).
MAF: Minor allele frequency in 190 Japanese Black sires.
GeneBank Accession Number: LOC523874 XM_602183; NCAPG NM_001102376; LOC540095 XM_001250262

Table 3 shows the primers used for the PCR.

TABLE 3

| | | |
|---|---|---|
| SNP ID | Forward primer (Seq ID No.) | Reverse primer (Seq ID No) |

| | | |
|---|---|---|
| SNP 1 | TGTACCTTGTGATACATGCTTTAAAAT(15) | GATCTGTACACAATAGGAGTTCAATAA(16) |
| SNP 2 | CACAGGGGAGTTGAATAGCAG(17) | CCTGTTGCTTCCAAGTAGACC(18) |
| SNP 3 | CAGAAGCAGCTGACACAGGA(19) | ACTCACAGACTGCTGCATCG(20) |
| SNP 4 | GGAGAAAACCCACAAGCTCA(21) | GCCTCCGAGACAAAGTTTCA(22) |
| SNP 5 | GGGATGTTGGCAGAAAAGAA(23) | CATGCCAAATATTTTTCAAAGG(24) |
| SNP 6 | TTGTAGATAATTTTCTTAGGTGAAGGA(25) | GGACACTCTTTCCTAAACCTTTT(26) |
| SNP 7 | TTCTCACTTAATGGGGAGCTG(27) | TTAGGAGAGCAAATTAGAACAAGAG(28) |
| SNP 8 | TTTCAGAATGTGAATTTTGGCTTA(29) | AGCCAAAAGCACTGAAAACAC(30) |
| SNP 9 | TTTCAGAATGTGAATTTTGGCTTA(31) | AGCCAAAAGCACTGAAAACAC(32) |
| SNP 10 | TGGATACTGTTTGGAGTTTTGTG(33) | TCAGTCGGGCACATACAGAA(34) |
| SNP 11 | TGGATACTGTTTGGAGTTTTGTG(35) | TCAGTCGGGCACATACAGAA(36) |
| SNP 12 | TTCTGTATGTGCCCGACTGA(37) | TCTGGCAGCTAAATTAAGCAAA(38) |
| SNP 13 | TTTACTTTTGGTGGGGGATG(39) | TGCTAAAAATGACCTTGCACA(40) |
| SNP 14 | GAGCTTACATGGGGAGGGTTA(41) | CTTCAAGAAATGAGCACCAAA(42) |
| SNP 15 | AGTATTTGGTGCTCATTTCTTGA(43) | TGAATTTAATTAGAAAAACTCTTCCAT(44) |
| SNP 16 | GCTGCTTTTGGGACTGATTG(45) | GCAGCAGCAAGACATTGAAA(46) |
| SNP 17 | TTTTAAGCTCAATGGAATCAGGA(47) | TGGAATCGCACACCAGAAAT(48) |
| SNP 18 | ATGGGGTACCTCACAGCACT(49) | AAGAAAACCTGAATCTTTTTCACC(50) |
| SNP 19 | CGCCGCTCGTATGTAAATG(51) | TGAACTGACCCGAAAGGAAG(52) |

First, 94 steers (up to 5 offspring from the same sire) in the highest carcass weight group (570-670 kg; top 4.7%) and 96 steers (up to 5 offspring from the same sire) in the lowest carcass weight group (290-410 kg; bottom 4.6%) among 7990 Japanese Black steers were genotyped and Fisher's exact test for 2×2 tables was performed (see “p-value” in Table 3). The results indicated the highest association of the e9 site with carcass weight (SNP 9 in Table 4: p(test using the number of alleles)=1.2×10⁻¹¹).

TABLE 4

| | p (test using the | p (test in | p (test in |
|---|---|---|---|
| SNP ID | number of alleles) | dominant model) | recessive model) |
| SNP 1 | 9.9E−05 | 2.1E−04 | 0.011 |
| SNP 2 | 1.0E−04 | 6.1E−06 | 0.0072 |
| SNP 3 | 4.4E−05 | 3.1E−04 | 0.0035 |
| SNP 4 | 0.0016 | 0.0030 | 0.091 |
| SNP 5 | 1.0 | ND | ND |
| SNP 6 | 1.0 | ND | ND |
| SNP 7 | 0.91 | ND | ND |
| SNP 8 | 0.82 | ND | ND |
| SNP 9 | 1.2E−11 | 6.7E−11 | 0.012 |
| SNP 10 | 0.0037 | 0.0032 | 0.20 |
| SNP 11 | 0.012 | 0.0066 | 0.16 |
| SNP 12 | 0.0067 | 0.0041 | 0.12 |
| SNP 13 | 1.0 | ND | ND |
| SNP 14 | 0.0016 | 0.0030 | 0.091 |
| SNP 15 | 0.82 | ND | ND |
| SNP 16 | 1.6E−10 | 6.1E−10 | 0.024 |
| SNP 17 | 0.83 | ND | ND |
| SNP 18 | 0.0091 | 0.0066 | 0.12 |
| SNP 19 | 0.0091 | 0.0066 | 0.12 |

ND: Since Sire A has homozygous alleles, the test was not performed.

Next, haplotypes consisting of the 19 SNPs were inferred using the fastPHASE program (Scheet, P. and M. Stephens (2006) Am J Hum Genet 78, 629-644). As a result, only the haplotype in which the e9 site was G was detected at a higher frequency in the highest carcass weight group than in the lowest carcass weight group (haplotypes 5 and 6 in Table 5: the p-value of Fisher's exact test using a 2×2 table for these haplotypes and the other haplotypes was p=6.7×10⁻¹¹).

TABLE 5

| | the | the | | | | | | | | | |
|---|---|---|---|---|---|---|---|---|---|---|---|
| | highest | lowest | US01 | | | | NCAPG | | | | |
| Haplotype | group | group | SNP 1 | SNP 2 | SNP 3 | SNP 4 | SNP 5 | SNP 6 | SNP 7 | SNP 8 | SNP 9 |
| 1 | 5 | 4 | C | A | G | C | G | 2 | A | A | T |
| 2 | 52 | 58 | G | G | A | C | G | 2 | A | A | T |
| 3 | 67 | 99 | C | A | G | C | A | 1 | G | C | T |
| 4 | 11 | 27 | C | A | G | G | A | 1 | G | C | T |
| 5 | 2 | 1 | C | A | G | G | A | 1 | G | C | G |
| 6 | 43 | 5 | G | G | A | G | A | 1 | G | C | G |
| [symbol] | 180 | 194 | | | | | | | | | |

| | NCAPG | | | | | | | | MLR1 | |
|---|---|---|---|---|---|---|---|---|---|---|
| Haplotype | SNP 10 | SNP 11 | SNP 12 | SNP 13 | SNP 14 | SNP 15 | SNP 16 | SNP 17 | SNP 18 | SNP 19 |
| 1 | C | T | T | T | C | A | 1 | G | C | 1 |
| 2 | C | T | T | T | C | A | 1 | G | C | 1 |
| 3 | C | A | C | C | C | G | 1 | A | T | 2 |
| 4 | A | T | T | C | A | G | 1 | A | C | 1 |
| 5 | A | T | T | C | A | G | 1 | A | C | 1 |
| 6 | A | T | T | C | A | G | 2 | A | C | 1 |
| [symbol] | | | | | | | | | | |

These findings indicate that the genomic variation affecting carcass weight is G at the e9 site of the NCAPG gene and that this is a dominant or additive mutation.

### (4) Use of the SNP as a Marker

The offspring of Sires A-D were genotyped and their association with carcass weight was examined. The results are shown in Tables 6 and 7.

TABLE 6

| | GG | | | GT | | | TT | | |
|---|---|---|---|---|---|---|---|---|---|
| | Off- | CW | | Off- | CW | | Off- | CW | |
| Family | spring | Ave. | SD | spring | Ave. | SD | spring | Ave. | SD |
| Sire A | 47 | ##### ± | 47.7 | 241 | ##### ± | 44.9 | 151 | ##### ± | 41.3 |
| Sire B | 60 | ##### ± | 34.8 | 166 | ##### ± | 48.7 | 112 | ##### ± | 44.5 |
| Sire C | 49 | ##### ± | 26.7 | 220 | ##### ± | 26.2 | 139 | ##### ± | 30.4 |
| Sire D | 37 | ##### ± | 41.8 | 128 | ##### ± | 46.5 | 79 | ##### ± | 46.4 |
| D (female) | 11 | ##### ± | 41.5 | 54 | ##### ± | 38.1 | 44 | ##### ± | 43.0 |
| Sire E | 54 | 473.6 ± | 31.4 | 119 | 450.0 ± | 42.9 | 59 | 431.9 ± | 34.7 |
| 375 | 18 | ##### ± | 51.9 | 106 | ##### ± | 48.6 | 251 | ##### ± | 46.5 |

All the offspring except Sire D (female offspring) are steers.

TABLE 7

| | p (t-test) | p (t-test) | freq. of | Contribution |
|---|---|---|---|---|
| Family | GG vs. GT | GT vs. TT | G allele | ratio (%) |
| Sire A | 0.17 | 3.7E−10 | 0.20 | 8.7 |
| Sire B | 0.070 | 3.3E−05 | 0.35 | 6.1 |
| Sire C | 0.027 | 3.9E−03 | 0.31 | 2.9 |
| Sire D | 0.34 | 7.5E−03 | 0.32 | 1.8 |
| D(female) | 0.17 | 1.1E−04 | 0.35 | 13.2 |
| Sire E | 4.1E−05 | 1.5E−03 | 0.37 | 11.5 |
| 375 | 0.22 | 1.7E−07 | 0.19 | 8.1 |

Contribution ratio: the proportion of the trait variance explained by the genotype in the total variance of the phenotypic values.

The effect of an increase in carcass weight judged by the SNP of G at the e9 site of the NCAPG gene was consistently exerted by the change in one allele (heterozygous individual). When both alleles are G (homozygous individuals), the average of carcass weight tended to be higher than that of heterozygous individuals, but difference was significant only in Sire C and Sire E families.

Further, since a similar result was obtained from genotyping of an arbitrary population consisting of 375 offspring, it can be concluded that this SNP can widely be utilized as an excellent marker with which genotypes causing increase in carcass weight can be selected.

1. A method for evaluating genetic ability for carcass weight in a bovine individual,
comprising determining the nucleotide at the e9 site of the NCAPG gene or the amino acid at the E9 site of the NCAPG protein.

2. A bovine NCAPG gene, comprising G at the e9 site.

3. A bovine NCAPG protein, comprising methionine at the E9 site.

4. A DNA, comprising a part or the whole of a bovine NCAPG gene containing the e9 site of the bovine NCAPG gene, wherein the nucleotide at the e9 site is G.

5. A gene marker used to evaluate genetic ability for carcass weight in a bovine individual, consisting of a DNA, comprising a part or the whole of a bovine NCAPG gene containing the e9 site of the bovine NCAPG gene.

6. A method for selecting abovine individual having a higher genetic ability for carcass weight, comprising steps of:
determining the nucleotide at the e9 site of an NCAPG gene in each bovine individual; and
selecting an individual in which the nucleotide is G in at least one of the alleles of the NCAPG gene.

7. A method for increasing genetic ability for carcass weight of a bovine individual,
comprising generating a bovine individual in which the nucleotide at the e9 site is substituted by G in at least one of the alleles of an NCAPG gene by gene recombination technology.

8. A method for increasing genetic ability for carcass weight of a bovine individual,
comprising generating a bovine individual expressing an NCAPG protein in which the amino acid at the E9 site is methionine by gene recombination technology.

9. A bovine individual, comprising an exogenous DNA encoding an NCAPG protein in which is the amino acid at the E9 site is methionine.

10. The bovine individual of claim 9, wherein the exogenous gene is an expression vector expressing the NCAPG protein.

11. An expression vector expressing an NCAPG protein in which the amino acid at the E9 site of the NCAPG protein is methionine.


SEQUENCE LISTING (54 sequences)

SEQ ID NO: 1 - DNA, 3231 bp, Bos taurus
ggtaggcgaacgtgaacaggctttgtctcggccgggtactggcgccatggggaaggagaagagactgctgctgattaaggaggccttccagctggcgcagcagcctcaccagaaccaggcgaagctggtggtggcgctgaaccgcacctacggctcggtggatgacaaaacagattttcatgaggagtttgttcattaccttaaatatgctatggtggtctataaacgagaaccagctgtggaaagagtaatagaatttgccgcaaagtttgttacttcatttcaccaatcagatatggaaaatgatgaagaggaggaggaggatggtggcattttaaattatttgcttacttttctattaaagtctcatgaagcaaacagcaatgcagttagatttagagcgtgccagctcataaacaagctcttgggaaatatgccagaaaatgcccaaattgatgatgatttgtttgataaaattaatgaagccatgcttattagattgaaagataaagttccaaatgtaaggatacaggcagttcttgctctttcacgccttcaggatcccaaagatgatgaatgcccagtggttaatgcatatgctactttgattgaaaatgattcaaatccagaagttaggcgggcagtgttatcgtgtattgcgccatcagcaaagactttgccaaaaattgttgggcgcaccaaggatgtgaaagaaactgtcagaaagctggcttatcaggttttagctgaaaaggttcacatgagagctctgtccattgctcagagagtaatgctccttcaacaaggtctcaatgaccgatcagatgctgtgaaacaagcaatgcagaagcatcttctccaaggctggttacgttttactgaaggaaatatattagagttgcttcatcgattggatgtggaaaattcttctgaagtagcagtctctgttctcaatgccttgttttccatgactcctcttaatgaactggcagaaatctgtaaaaataatgacggcaggaaattgattccagcagatacattaactcctgaatttgctttgtattggcgtgtcctttgtgaacatttgaaatcaaaaggagaagaaggtgaagaatttttagagcagattttgccagagcctgtagtatatgcagagtatttactgagttatattcaaagcattccagttgttactgaagaacagagaggtgatttttcctatattggcaatttgatgacaaaagaattcataggtcaacaattaattctaattatcaagtctttggataccaatgaagaaggaggaaggaaacgaatactgggtatcttacaggagattcttactctacctaccacaccaatatccctaatttcttttcttgttgagagactgctccacatcattatagatgataataagagaatacaaattgttacagaaattatctcagagattcgggcacccattgttactgttgctgttaataatgatccagctgatgcaagaaagaaagagcttaagatggccgaaataaaagttaaacttattgaggcaaaagactctttggaaaattgcattaccttacaggattttcatcgagcatcagaattaaaagaagaaataaaagcattagaggatgccaaaataaaccttttgaaagagacagagcaacatgaaatgaaagaagtccacatagagaagaatgatgctgaaaccctacagaagtgtcttattttatgctatgaactattgaagcagatgtccacttcaacaggtataggtgcaaccatggatggcatcattgaatctttgattcttcctggaataataaatgttcatcctgtagtaagaaatttggctgtactgtgtttgggatgctgtggactgcagaatcaggattttgcaagtaaacactttgtattactcttgcaggttttgcaaattgatgatgtgacaataaaaataagtgctttaaaggcaatctttgaccaactgatgacatttggatttgaaccatttaaaactaaaaaaatcaaagctactcaaaaggaaggtgcagaaataaactccagtgaagagcaagagtcaaaagaatccgaagaagagacagctatagccaagaatgttctgaaactactttccgatttcttagatagtgaggtgtctgaactcagaacaggagctgcagaaggactagccaagctgatgttctctggacttttggtcagcagcaggattctttctcatcttgtcttgttatggtacaaccctgtgactgaagaggacattcgacttcgacattgcctcggcgtgttcttccccatgtttgcttatgcaagcaggactaaccaggaatgttttgaagaagcctttcttccaactctgcaaacactggccaatgcccctgcgtcatctcctctagctgaaatagatataactaatgttgctgagttacttgtagatttgacaagaccaagtgggttaaatcctcaggccaagaatcccccagattatcaggccttaacagttcatgacaatctggctatgaaaatttgcaatgagatcctaacatgtccacattcaccagaagttcgggtctatacgaaagctttgagttctttagaactcagcagcgatcttgctaaagatcttctggttgtgctgaatgagattctggagcaagtaaaagatagaacatgtctaagagctctggagaaaatcaagattcagatagaaaaaggaattaaagaacatagtgaccaagctgtagcagcacaggatgacatcacaactatgactgttcttcagagtgaagatgaaaagaataaagatgtatacataactcctgtcaaggaagtaaaagcaactcgaatgaaatccactcagcaaaagaccaacagaggacggagaaaagtggtagcttcagctagaacgaacagaagatgtcagactattgaagctgaggctaactctgaaagtgatcatgaagttccagaaccagaatcagaaatgaagatgagattaccaagacgagccaaaacagcagcactagaaaaaagtaaacttaaccttgcacaatttctcaatgaagatacaagttaggagaagaaatgatggaggtggagtcctttgaaaaatggcctttaaaattatgttcagttctttgctttaataaagttacccttgtatgaaaattaaagtctgattcttgcagaaaaaaaaaaaaaaaa

SEQ ID NO: 2 - protein, 1018 aa, Bos taurus
MGKEKRLLLIKEAFQLAQQPHQNQAKLVVALNRTYGSVDDKTDFHEEFVHYLKYAMVVYKREPAVERVIEFAAKFVTSFHQSDMENDEEEEEDGGILNYLLTFLLKSHEANSNAVRFRACQLINKLLGNMPENAQIDDDLFDKINEAMLIRLKDKVPNVRIQAVLALSRLQDPKDDECPVVNAYATLIENDSNPEVRRAVLSCIAPSAKTLPKIVGRTKDVKETVRKLAYQVLAEKVHMRALSIAQRVMLLQQGLNDRSDAVKQAMQKHLLQGWLRFTEGNILELLHRLDVENSSEVAVSVLNALFSMTPLNELAEICKNNDGRKLIPADTLTPEFALYWRVLCEHLKSKGEEGEEFLEQILPEPVVYAEYLLSYIQSIPVVTEEQRGDFSYIGNLMTKEFIGQQLILIIKSLDTNEEGGRKRILGILQEILTLPTTPISLISFLVERLLHIIIDDNKRIQIVTEIISEIRAPIVTVAVNNDPADARKKELKMAEIKVKLIEAKDSLENCITLQDFHRASELKEEIKALEDAKINLLKETEQHEMKEVHIEKNDAETLQKCLILCYELLKQMSTSTGIGATMDGIIESLILPGIINVHPVVRNLAVLCLGCCGLQNQDFASKHFVLLLQVLQIDDVTIKISALKAIFDQLMTFGFEPFKTKKIKATQKEGAEINSSEEQESKESEEETAIAKNVLKLLSDFLDSEVSELRTGAAEGLAKLMFSGLLVSSRILSHLVLLWYNPVTEEDIRLRHCLGVFFPMFAYASRTNQECFEEAFLPTLQTLANAPASSPLAEIDITNVAELLVDLTRPSGLNPQAKNPPDYQALTVHDNLAMKICNEILTCPHSPEVRVYTKALSSLELSSDLAKDLLVVLNEILEQVKDRTCLRALEKIKIQIEKGIKEHSDQAVAAQDDITTMTVLQSEDEKNKDVYITPVKEVKATRMKSTQQKTNRGRRKVVASARTNRRCQTIEAEANSESDHEVPEPESEMKMRLPRRAKTAALEKSKLNLAQFLNEDTS

SEQ ID NO: 3 - DNA, 20 bp, Bos taurus
agccagctgagtcaaattcc

SEQ ID NO: 4 - DNA, 20 bp, Bos taurus
gtgagacagatgggcaatca

SEQ ID NO: 5 - DNA, 21 bp, Bos taurus
tcagcttctgtacccatggac

SEQ ID NO: 6 - DNA, 20 bp, Bos taurus
agccagggtttccagaaaag

SEQ ID NO: 7 - DNA, 20 bp, Bos taurus
cctttgtttgctgggtcaat

SEQ ID NO: 8 - DNA, 20 bp, Bos taurus
gggcttgatctctggttgag

SEQ ID NO: 9 - DNA, 20 bp, Bos taurus
atggcaacccactactccag

SEQ ID NO: 10 - DNA, 20 bp, Bos taurus
ttgctaccaagcaagcactg

SEQ ID NO: 11 - DNA, 19 bp, Bos taurus
gtaaactcaagccacggca

SEQ ID NO: 12 - DNA, 21 bp, Bos taurus
cgacaaccttgatgtgacaaa

SEQ ID NO: 13 - DNA, 20 bp, Bos taurus
gatggcactggaggtagagc

SEQ ID NO: 14 - DNA, 20 bp, Bos taurus
caaccccatggattgtaacc

SEQ ID NO: 15 - DNA, 27 bp, Bos taurus
tgtaccttgtgatacatgctttaaaat

SEQ ID NO: 16 - DNA, 27 bp, Bos taurus
gatctgtacacaataggagttcaataa

SEQ ID NO: 17 - DNA, 21 bp, Bos taurus
cacaggggagttgaatagcag

SEQ ID NO: 18 - DNA, 21 bp, Bos taurus
cctgttgcttccaagtagacc

SEQ ID NO: 19 - DNA, 20 bp, Bos taurus
cagaagcagctgacacagga

SEQ ID NO: 20 - DNA, 20 bp, Bos taurus
actcacagactgctgcatcg

SEQ ID NO: 21 - DNA, 20 bp, Bos taurus
ggagaaaacccacaagctca

SEQ ID NO: 22 - DNA, 20 bp, Bos taurus
gcctccgagacaaagtttca

SEQ ID NO: 23 - DNA, 20 bp, Bos taurus
gggatgttggcagaaaagaa

SEQ ID NO: 24 - DNA, 22 bp, Bos taurus
catgccaaatatttttcaaagg

SEQ ID NO: 25 - DNA, 27 bp, Bos taurus
ttgtagataattttcttaggtgaagga

SEQ ID NO: 26 - DNA, 23 bp, Bos taurus
ggacactctttcctaaacctttt

SEQ ID NO: 27 - DNA, 21 bp, Bos taurus
ttctcacttaatggggagctg

SEQ ID NO: 28 - DNA, 25 bp, Bos taurus
ttaggagagcaaattagaacaagag

SEQ ID NO: 29 - DNA, 24 bp, Bos taurus
tttcagaatgtgaattttggctta

SEQ ID NO: 30 - DNA, 21 bp, Bos taurus
agccaaaagcactgaaaacac

SEQ ID NO: 31 - DNA, 24 bp, Bos taurus
tttcagaatgtgaattttggctta

SEQ ID NO: 32 - DNA, 21 bp, Bos taurus
agccaaaagcactgaaaacac

SEQ ID NO: 33 - DNA, 23 bp, Bos taurus
tggatactgtttggagttttgtg

SEQ ID NO: 34 - DNA, 20 bp, Bos taurus
tcagtcgggcacatacagaa

SEQ ID NO: 35 - DNA, 23 bp, Bos taurus
tggatactgtttggagttttgtg

SEQ ID NO: 36 - DNA, 20 bp, Bos taurus
tcagtcgggcacatacagaa

SEQ ID NO: 37 - DNA, 20 bp, Bos taurus
ttctgtatgtgcccgactga

SEQ ID NO: 38 - DNA, 22 bp, Bos taurus
tctggcagctaaattaagcaaa

SEQ ID NO: 39 - DNA, 20 bp, Bos taurus
tttacttttggtgggggatg

SEQ ID NO: 40 - DNA, 21 bp, Bos taurus
tgctaaaaatgaccttgcaca

SEQ ID NO: 41 - DNA, 21 bp, Bos taurus
gagcttacatggggagggtta

SEQ ID NO: 42 - DNA, 21 bp, Bos taurus
cttcaagaaatgagcaccaaa

SEQ ID NO: 43 - DNA, 23 bp, Bos taurus
agtatttggtgctcatttcttga

SEQ ID NO: 44 - DNA, 27 bp, Bos taurus
tgaatttaattagaaaaactcttccat

SEQ ID NO: 45 - DNA, 20 bp, Bos taurus
gctgcttttgggactgattg

SEQ ID NO: 46 - DNA, 20 bp, Bos taurus
gcagcagcaagacattgaaa

SEQ ID NO: 47 - DNA, 23 bp, Bos taurus
ttttaagctcaatggaatcagga

SEQ ID NO: 48 - DNA, 20 bp, Bos taurus
tggaatcgcacaccagaaat

SEQ ID NO: 49 - DNA, 20 bp, Bos taurus
atggggtacctcacagcact

SEQ ID NO: 50 - DNA, 24 bp, Bos taurus
aagaaaacctgaatctttttcacc

SEQ ID NO: 51 - DNA, 19 bp, Bos taurus
cgccgctcgtatgtaaatg

SEQ ID NO: 52 - DNA, 20 bp, Bos taurus
tgaactgacccgaaaggaag

SEQ ID NO: 53 - DNA, 21 bp, Bos taurus
caccatgtcctgacctcagat

SEQ ID NO: 54 - DNA, 20 bp, Bos taurus
taacagtgccctgcatgaga